(19) United States
(12) Patent Application Publication
(10) Pub. No.: US 2007/0053950 A1
(43) Pub. Date: Mar. 8, 2007

(54) Composition of polymer microcapsules of biocide for coating material

(76) Inventors: Shukla Parshuram Gajanan, Pune (IN); Swaminathan Sivaram, Pune (IN); Ahmad Absar, Pune (IN)

Correspondence Address: LUEDEKA, NEELY & GRAHAM, P.C., P O BOX 1871, KNOXVILLE, TN, 37901, US

(21) Appl. No.: 11/361,551

(22) Filed: Feb. 24, 2006

(30) Foreign Application Priority Data
Feb. 28, 2005 (IN) 425/DEL/2005

(51) Int. Cl. A01N 25/34; A61K 9/48

(52) U.S. Cl. 424/408; 424/451

(57) ABSTRACT

The invention provides composition comprising microcapsules containing biocides for coating material such as paint. The invention describes composition of microcapsules containing biocides, especially Irgarol (algaecide) and Zinc Pyrithione (fungicide) and coating material such as paint to increase the life of coating material from the attack of algae and fungi respectively. The invention demonstrates that microcapsules containing biocide dispersed in paint show 0 to 3% reduction in inhibition zone where as un-encapsulated biocide show 20-22% reduction in inhibition zone when analyzed by filter paper assay thus indicating extended duration of biocidal activity with microencapsulated biocide.

### FIELD OF THE INVENTION

This invention relates to a composition of microcapsules containing biocide for coating material. More, particularly the present invention relates to a composition comprising of microcapsules containing biocide for coating material such as paint. The invention describes composition typically illustrated by composition comprising microcapsules containing biocides, especially Irgarol (algaecide) and Zinc Pyrithione (fungicide) and coating material such as paint to increase the life of coating material from the attack of algae and fungi respectively. The invention demonstrates that microcapsules containing biocide dispersed in paint show 0 to 3% reduction in inhibition zone where as un-encapsulated biocide show 20-22% reduction in inhibition zone when analyzed by filter paper assay thus indicating extended duration of biocidal activity with microencapsulated biocide.

### BACKGROUND OF THE INVENTION

Biocides are chemical compounds, which are toxic to microbial cells and are added to different types of products to prevent the growth of unwanted microorganisms. Paint is one of such products. Paint applied for exterior or interior use typically has two basic functions, protection and decoration. Attack of fungi and algae either in wet state or at the site of application of paint can destroy these functions and thus is one of the major factors responsible for reduction in the life of paint film. Paint films can be made fungi- algae- and bacteria-free by incorporating suitable biocide in the paint formulation. Reduction in biocidal activity of paint film is mostly attributed to the factors such as chemical degradation of biocide, fast dissipation of biocide from paint film due to washing out and/or volatilization from the paint film. The life of paint film will be more if these biocides are retained in the film and on the film surface for longer period. This extended duration of biocidal activity can be achieved by incorporating biocide in Controlled Release (CR) form. Microcapsule is one of the best controlled release form, wherein an active agent (core material) is surrounded by a polymer film or uniformly dispersed in a polymer matrix.

### Controlled Release Biocides

Controlled Release (CR) biocides has been the subject of interest to many researchers. CR concept and work was initiated first time on antifouling paints using chloroprene polymer by N. F. Cardarelli (Cardarelli N. F., Chapter 3 In Controlled Release Technologies: Methods, Theory & Applications, Ed. Kydonieus A. F., 1980, CRC Press Inc., USA). Antifouling marine paint composition containing gelation microcapsules of water-immiscible biocide has been described in U.S. Pat. No. 4,253,877. Yet another U.S. Pat. No. 5,378,413 describes preparation of gelatin microcapsules containing fouling reducing agents and their use in paint system. Another report describes extended control of marine fouling using formulation of microencapsulated organometallic biocide and vinyl rosin paint. (Porter R. and Miale J. B., Appl. Biochem. and Biotech., 9 (1984), p 439-445 (CA 102:162052).

Apart from CR antifouling formulations there have been very few reports on CR of other biocides. Biocide namely 4,5-dichloro-n-octyl-3-isothiazolinone (DCOI) can be encapsulated in a variety of polysiloxane matrices using sol-gel chemistry (Ghosh T. and Nungesser E. N., Proc. Int. Symp. Control. Rel. Bioact. Mater., 25 (1998), p 324 ). The skin sensitization potential of active agent (3-isothiazolone) in loci such as water-based marine antifouling paint of decorative is reduced by encapsulating the active agent in polyurea (.EP 679333 (1995), (CA 123:332738)). The fungicide tebuconazole and chlorothalonil were successfully incorporated into polyvinylpyridine (PVPy) and polyvinylpyridine-co-styrene nano particles (Liu Y. et.al., J. Appl. Poly. Sci, 79 (2001), p 458-465). U.S. Pat. (No. 4915947) describes preparation of microencapsulated phytotoxic fungicides using crosslinked polyurea or polyamide to provide an effective agent for direct foliar application to control fungal diseases on crops. Urea—formaldehyde (UF) and/or melamine formaldehyde (MF) resins have been used to prepare microcapsules of fungicide namely 3-Iodo-2-propynylbutyl carbamate. These microcapsules when incorporated into exterior latex paint and applied onto rubberwood panels on exposing to the environment showed longer protection from discoloration. (Ibrahim W. A. et.al., Pertanika 12 (1989) p 409-412 (CA 114:25832)). The acrylic latex exterior paint containing microcapsules of fungicides 2,3,5,6-tetrachloro-4-methylsulfonylpryidine and tetrachloroisophthao nitrile have been reported to show good mildew protection (Noren G. K. et.al., J. Coatings Tech. 58 (1986), p 31-3⁹ (CA 104: 188225)).

Another patent describes encapsulation of biocide using MF resin and their use in coating material like plaster having silicate, mineral or polymer resin binder or a primer based on a silicate or polymer resin binder (Patent WO 2004000953 A1 20031231 (CA 140:61138)).

Biocides play an important role in paint formulations. However reduction in biocidal activity due to factors such as chemical degradation of biocide and fast dissipation of biocide due to washing out from the paint film, is a problem which leads to decrease in life period of paint. To compensate the dissipation of biocide use of excess amount of biocide in the coating material composition leads to environmental pollution risk.

In the prior art there neither exists microcapsules of biocides like Irgarol and Zinc Pyrithione in particular (except as reported in our co-pending patent application no. 448/INF/2004 and 449/NF/2004) nor any composition describing use of microcapsules of these biocides.

Thus to avoid reduction in activity of biocides due to factors mentioned above and to satisfy the need to prolong the life of biocide and thus coating material like paint, present invention provides coating material composition comprising microencapsulated biocide which is more effective than composition comprising un-encapsulated biocide.

### OBJECTIVES OF THE INVENTION

The main object of the present invention is to provide a composition comprising microencapsulated biocide and coating material such as paint.

Another object of the present invention is to demonstrate that coating material composition comprising microencapsulated biocide is more effective than composition comprising un-encapsulated biocide and an increase in protection from fungus or algae is obtained with microencapsulated biocide as compared to un-encapsulated biocide.

### SUMMARY OF THE INVENTION

Accordingly the present invention provides a composition of polymer microcapsules of biocide for coating material, the said composition comprising polymer microcapsules of biocide in the range of 2-20 wt % of the coating material.

In an embodiment of the present invention composition the encapsulating polymer used in polymer microcapsules is selected from the group consisting of poly(methyl methacrylate), polystyrene, poly(vinylpyridine-co-styrene), polyamide, polyester, ethyl cellulose and polyurethane.

In yet another embodiment the biocide encapsulated in polymer microcapsules is selected from Zinc Pyrithione and Irgarol.

In yet another embodiment the amount of biocide used in polymer microcapsules of biocide is in the range of 20-70 wt % based on total weight of microcapsules.

In yet another embodiment the particle size of polymer microcapsules of biocide used is in the range of 1-100 microns.

In yet another embodiment the coating material used is paint.

In yet another embodiment the encapsulating polymer used in polymer microcapsule optionally contain a crosslinking agent.

In yet another embodiment the crosslinking agent used in encapsulating polymer is selected from the group consisting of trimethylol propane, glycerol and hexane triols.

In yet another embodiment the amount of crosslinking agent used in encapsulating polymer is in the range 1 to 10 wt %.

In yet another embodiment the composition is useful to increase the life of coating material from the attack of undesired microorganisms such as algae and fungi.

In still another embodiment the composition shows biocidal activity against fungal strain Aspergillus niger (NRRL337) and algal strains Oscillatoria tenius and Chlorella fusca.

In a feature of the present invention the use of microcapsules in coating material composition are not to be restricted to polyurethane or polystyrene or poly (methyl methacrylate) microcapsules containing biocides Zinc Pyrithione or Irgarol but may be selected from other class of polymer microcapsules containing other different biocides.

In a feature the uses of microcapsules are not restricted to the above said fungal and algal strain but may be used for other fungal and algal strains.

### DETAIL DESCRIPTION OF THE INVENTION

The invention provides composition comprising microcapsules containing biocides for coating material such as paint. The invention describes composition of microcapsules containing biocides, especially Irgarol (algaecide) and Zinc Pyrithione (fungicide) and coating material such as paint to increase the life of coating material from the attack of algae and fungi respectively. The invention demonstrates that microcapsules containing biocide dispersed in paint show 0 to 3% reduction in inhibition zone where as un-encapsulated biocide show 20-22% reduction in inhibition zone when analyzed by filter paper assay thus indicating extended duration of biocidal activity with microencapsulated biocide.

The polymer microcapsules of biocide and a process for the preparation thereof has been claimed and described in our co-pending Indian patent applications 448/NF/2004 and 449/NF/2004.

The invention is further illustrated by the following examples which should not be construed to limit the scope of the invention.

### EXAMPLE 1

1 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 3 g is dispersed in this surfactant solution followed by addition of 0.5 g DBTDL catalyst solution (1.0 w/w % solution in paraffin oil) and 2 g of ethylene glycol and the mixture is agitated at 1000 rotations per minute using turbine type stirrer. To this mixture 0.1 g of fumed silica is added. After 15-30 minutes, 6.74 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 11.3 g. Microcapsules have particle size range of 1-45 microns of which a majority of particles are 20-25 microns.

### EXAMPLE 2

1.28 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 14 g is dispersed in this surfactant solution followed by addition of 0.5 g DBTDL catalyst solution (1.0 w/w % solution in paraffin oil) and 2 g of ethylene glycol and the mixture is agitated at 1000 rotations per minute using turbine type stirrer. To this mixture 0.1 g of fumed silica is added. After 15-30 minutes, 6.74 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 21.1 g. Microcapsules have particle size range of 5-50 microns of which a majority of particles are 10-20 microns.

### EXAMPLE 3

1.3 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 14 g is dispersed in this surfactant solution followed by addition of 0.5 g DBTDL catalyst solution (1.0 wt % solution in paraffin oil) and 2 g of ethylene glycol containing 0.2 g of trimethylol propane and the mixture is agitated at 1000 rotations per minute using turbine type stirrer. To this mixture 0.1 g of fumed silica is added. After 15-30 minutes, 6.99 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 22.4 g. Microcapsules have particle size range of 5-50 microns of which a majority of particles are 10-20 microns.

### EXAMPLE 4

1.3 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 14 g is dispersed in this surfactant solution followed by addition of 0.5 g DBTDL catalyst solution (1.0 wt % solution in paraffin oil) and 2 g of ethylene glycol containing 0.4 g of trimethylol propane and the mixture is agitated at 1000 rotations per minute using turbine type stirrer. To this mixture 0.1 g of fumed silica is added. After 15-30 minutes, 7.46 g of toluene diisocyanate is added drop wise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 23.0 g. Microcapsules have particle size range of 5-45 microns of which a majority of particles are 5-20 microns.

### EXAMPLE 5

2.9 g of polymeric surfactant Hypermer A60 was dissolved in 10 g of paraffin oil by heating the mixture at 65° C. In a jacketed reaction kettle having volume of 250 mL. 90 g of paraffin oil is taken followed by addition of above said surfactant solution at 25-27° C. Biocide namely Zinc Pyrithione 28 g is dispersed in this surfactant solution followed by addition of 1.0 g DBTDL catalyst solution (1.0 wt % solution in paraffin oil) and 4 g of ethylene glycol and the mixture is agitated at 1000 rotations per minute using turbine type stirrer. To this mixture 0.2 g of fumed silica is added. After 15-30 minutes, 13.5 g of toluene diisocyanate is added dropwise over a period of 20 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 44.9 g. Microcapsules have particle size range of 2-40 microns of which a majority of particles are 5-15 microns.

### EXAMPLE 6

0.82 g of polymeric surfactant Uniqema was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 7.3 g is dispersed in this surfactant solution followed by addition of 0.5 g DBTDL catalyst solution (1.0 wt % solution in paraffin oil) and 3 g of 2-ethyl 1,3 hexane glycol and the mixture is agitated at 1000 rotations per minute using turbine type stirrer. To this mixture 0.1 g of fumed silica is added. After 15-30 minutes, 4.28 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 13.3 g. Microcapsules have particle size range of 5-40 microns of which a majority of particles are 10-25 microns.

### EXAMPLE 7

2.98 g of polymeric surfactant Uniqema (HLB 6) was dissolved in 10 g of paraffin oil by heating the mixture at 65° C. In a jacketed reaction kettle having volume of 250 mL. 90 g of paraffin oil is taken followed by addition of above said surfactant solution at 25-27° C. Biocide namely Irgarol 17.5 g is dispersed in this surfactant solution followed by addition of 1.0 g DBTDL catalyst solution (1.0 wt % solution in paraffin oil) and 4 g of ethylene glycol and the mixture is agitated at 1000 rotations per minute using turbine type stirrer. To this mixture 0.1 g of fumed silica is added. After 15-30 minutes, 13.5 g of toluene diisocyanate is added drop-wise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 34.7 g. Microcapsules have particle size range of 2-50 microns of which a majority of particles are 5-20 microns.

### EXAMPLE 8

Polymeric surfactant namely poly(lauryl methacrylate)-g-poly(ethylene oxide) (number average molecular weight 34170, weight average molecular weight 96560) is synthesized as similar to procedure described in the literature (Palaskar D. V. et.al., Proc. of International Seminar on Frontiers of Polymer Science and Engineering, Macro 2002, held at Khargpur, India, Dec. 9-11, 2002). 1.17 g of this surfactant was dissolved in 5 g of paraffin oil by heating the mixture at 60° C. In a jacketed reaction kettle having volume of 250 mL. 45 g of paraffin oil is taken followed by addition of above said surfactant solution at 25-27° C. Biocide namely Irgarol 14.0 g is dispersed in this surfactant solution followed by addition of 0.5 g DBTDL catalyst solution (1.0 wt % solution in paraffin oil) and 2 g of ethylene glycol and the mixture is agitated at 1000 rotations per minute using turbine type stirrer. To this mixture 0.1 g of fumed silica is added. After 15-30 minutes, 6.85 g of toluene diisocyanate is added drop wise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 21.2 g. Microcapsules have particle size range of 2-60 microns of which a majority of particles are 5-20 microns.

### EXAMPLE 9

### Evaluation of Biocidal Activity of Composition Comprising Microencapsulated Biocide and Paint

To demonstrate that biocide can extend duration of activity through microencapsulation, the antifungal activity of microcapsules containing Zinc Pyrithione and unencapsulated Zinc Pyrithione against fungal strain Aspergillus niger (NRRL337) is assessed by filter paper bioassay by method reported elsewhere (Bauer, A. W. et.al., Amer. J. Clin. Pathol., 45 (1966) p 493-496 and Thomberry, H. H. Phytopathology 40 (1950) p 419-420). Microcapsules of Zinc Pyrithione and unencpasulated Zinc Pyrithione are dispersed in paint such that the concentration of Zinc Pyrithione is around 5 wt % based on weight of paint and taken for analysis after 0 days, 12 days and 30 days of dispersion period. The antifungal activity of all the composition samples is indicated by zone of inhibition that is developed around the filter paper discs against the vegetative growth after the spore germination. The zone of inhibition is measured after 2 days and 100 days after the day on which samples are taken for filter paper bioassay. Table 1 shows measurements of inhibition zones after 2 days and 100 days, obtained for various samples taken for analysis after 0 days (the day on which microcapsules are dispersed in paint). Microencapsulated Zinc Pyrithione samples show very less % reduction in inhibition zone as compared to unencapsulated Zinc Pyrithione demonstrating that extended duration of activity of biocide is achieved through microencapsulation. Similar type of results are obtained when microcapsules stored in paint for 12 days and 30 days are taken for such biocidal analysis.

TABLE 1

| | | Zone of | Zone of | % Reduction |
|---|---|---|---|---|
| Description | % of | inhibition | inhibition | in inhibition |
| of microcapsules | biocide | (mm) | (mm) | zone C = |
| (encapsulating | in micro- | (after 2 | (after 100 | [(A − B) × |
| polymer) | capsules | days) A | days) B | 100]/A |
| Unencapsulated | — | 41 | 32 | 21.9 |
| Zinc Pyrithione | | | | |
| Polyurethane | 60 | 40 | 40 | 0.0 |
| with 2.3% cross- | | | | |
| linking agent | | | | |
| Polyurethane | 60 | 37 | 36 | 2.70 |
| Polyurethane | 25 | 35 | 34 | 2.85 |
| PMMA | 28.8 | 39 | 38 | 2.56 |
| Polystyrene | 44.4 | 36 | 36 | 0 |
| Polystyrene | 28.8 | 36 | 35 | 2.78 |

The antialgal activity of microencapsulated Irgarol and unencapsulated Irgarol against algal strains (Oscillatoria tenius and Chlorella fusca) is assessed by filter paper bioassay by method as described above for fungal activity. Unencpasulated Irgarol and microcapsules of Irgarol are dispersed in paint such that the concentration of Irgarol is around 5 wt % based on weight of paint and taken for analysis after 0 days, 15 days and 30 days of dispersion period. Both the samples such as unencapsulated irgarol and microencapsulated irgarol dispersed in paint show very strong inhibitory activity against both the algal strains even after 100 days after the day on which samples are taken for filter paper bioassay.

### ADVANTAGES

Biocides play an important role in paint formulations. However reduction in biocidal activity due to factors such as chemical degradation of biocide and fast dissipation of biocide due to washing out from the paint film, is a problem which leads to decrease in life period of paint. To compensate the dissipation of biocide use of excess amount of biocide in the coating material composition leads to environmental pollution risk.

Thus to avoid reduction in activity of biocides due to factors mentioned above and to satisfy the need to prolong the life of biocide and thus coating material like paint, present invention provides coating material composition comprising microencapsulated biocide which is more effective than composition comprising un-encapsulated biocide.

We claim:

1. A composition of polymer microcapsules of biocide for coating material, the said composition comprising polymer microcapsules of biocide in the range of 2-20 wt % of the coating material.

2. A composition as claimed in claim 1, wherein the encapsulating polymer used in polymer microcapsules is selected from the group consisting of poly(methyl methacrylate), polystyrene, poly(vinylpyridine-co-styrene), polyamide, polyester, ethyl cellulose and polyurethane.

3. A composition as claimed in claim 1, wherein the biocide encapsulated in polymer microcapsules is selected from Zinc Pyrithione and Irgarol.

4. A composition as claimed in claim 1, wherein the amount of biocide used in polymer microcapsules of biocide is in the range of 20-70 wt % based on total weight of microcapsules.

5. A composition as claimed in claim 1, wherein the particle size of polymer microcapsules of biocide used is in the range of 1-100 microns.

6. A composition as claimed in claim 1, wherein the coating material used is paint.

7. A composition as claimed in claim 1 wherein the encapsulating polymer used in polymer microcapsule optionally contain a crosslinking agent.

8. A composition as claimed in claim 1, wherein the crosslinking agent used in encapsulating polymer is selected from the group consisting of trimethylol propane, glycerol and hexane triols.

9. A composition as claimed in claim 1, wherein the amount of crosslinking agent used in encapsulating polymer is in the range 1 to 10 wt %.

10. A composition as claimed in claim 1 is useful to increase the life of coating material from the attack of undesired microorganisms such as algae and fungi.

11. A composition as claimed in claim 1 shows biocidal activity against fungal strain Aspergillus niger (NRRL337) and algal strains Oscillatoria tenius and Chlorella fusca.
